(19) United States
(12) Patent Application Publication
(10) Pub. No.: US 2005/0053631 A1
(43) Pub. Date: Mar. 10, 2005

(54) Method of decreasing sebum production

(75) Inventors: Alexandra Draganescu D'Arcangelis, Randolph, NJ (US); John Steven Bajor, Ramsey, NJ (US); Kimberly Marie Campbell, North Bergen, NJ (US); Helen Elizabeth Knaggs, Weehawken, NJ (US)

Correspondence Address: UNILEVER INTELLECTUAL PROPERTY GROUP, 700 SYLVAN AVENUE,, BLDG C2 SOUTH, ENGLEWOOD CLIFFS, NJ, 07632-3100, US

(73) Assignee: Unilever Home & Personal Care USA, Division of Conopco, Inc.

(21) Appl. No.: 10/658,876

(22) Filed: Sep. 10, 2003

(51) Int. Cl. A61K 7/00

(52) U.S. Cl. 424/401

(57) ABSTRACT

Cosmetic compositions and methods of decreasing sebum production by contacting sebocytes with a fatty acid synthase inhibitor, including cerulenin, triclosan, α-methylene-γ-butyrolactone, and EGCG, as well as mixtures thereof and analogs thereof. The invention further relates to a cosmetic method of identifying sebum production inhibiting actives comprising contacting the active with FAS and detecting a reduction in sebum amount production in sebocytes.

### BACKGROUND OF THE INVENTION

1. Field of the Invention

The present invention relates to a method of decreasing sebum production in sebocytes. More specifically, the present invention relates to a method of decreasing sebum production by contacting sebocytes with a fatty acid synthase inhibitor.

2. Description of the Related Art

Sebum is skin oil which is produced by sebocytes (cells of the sebaceous glands in the skin) and is then secreted to the skin surface. A frequent and undesirable skin condition is “oily skin,” the condition which results from the excessive amount of sebum on the skin. Oily skin is associated with a shiny, undesirable appearance and a disagreeable tactile sensation and affects various age groups. Excessive sebum production is cosmetically undesirable and has been associated with the skin condition acne. Therefore, cosmetic products and methods that provide sebum control are highly desirable.

Sebum is made up of a number of components, with about 57% being triglycerides, or fatty acids. About 12% of sebum is squalene, about 3% of sebum are sterol esters, about 25% wax esters, and about 1 to about 2% cholesterol.

Fatty acid synthesis is catalyzed by an enzyme known as fatty acid synthase (FAS), one of four major enzymes comprising the fatty acid biosynthetic pathway in humans. The fatty acid biosynthetic pathway components include: acetyl-CoA carboxylase, which is the rate limiting enzyme that synthesizes malonyl-CoA; malic enzyme, which produces NADPH; citrate lyase, which synthesizes acetyl-CoA; and FAS, which catalyzes NADPH-dependent synthesis of fatty acids from acetyl-CoA and malonyl-CoA. Free fatty acids, the final products of FAS activity, require separate enzymatic derivatization with coenzyme-A for incorporation into other products. In higher organisms FAS is a multifunctional enzyme which is well known to carry out the following seven enzymatic functions on a single molecule:
(1) acetyl transacylase (AT),
(2) malonyl transacylase (MT),
(3) β-ketoacyl synthetase (the condensing enzyme),
(4) β-hydroxyacyl reductase,
(5) β-hydroxyacyl dehydrase (DH),
(6) enoyl reductase (ER), and
(7) thioesterase (TE).

Inhibition of FAS interferes with fatty acid synthesis and is disclosed, for example, in Kuhajda, U.S. Pat. No. 5,981,575 and Orlow, et al., PCT Patent Application WO 02/087565. However, no literature prior to the present invention has identified FAS as a target enzyme for reducing sebum production in sebocytes.

The present invention is based on the unexpected discovery that inhibition of FAS inhibits, reduces, or controls sebum production.

### SUMMARY OF THE INVENTION

It has surprisingly been discovered that various agents that inhibit fatty acid biosynthesis are useful for inhibiting sebum production. More specifically, it has been determined that agents capable of inhibiting mammalian fatty acid synthase (FAS) inhibit the production of sebum in sebocytes. These discoveries have been utilized to provide the present invention, which includes cosmetic methods and compositions useful for decreasing sebum production in skin.

In one aspect, the invention provides a method of decreasing sebum synthesis in a sebocyte or in skin of an individual in need thereof. The method comprises contacting the sebocyte with a sebum production inhibiting amount of FAS inhibitor, thereby reducing sebum synthesis in the sebocyte. The inventive method reduces synthesis of squalene, triglycerides, free fatty acids, wax esters, cholesterol, sterol esters, and/or a combination thereof.

In another aspect, the invention provides a method of identifying sebum production inhibiting actives comprising contacting the active with FAS and detecting a reduction in sebum amount production.

The invention also provides, in another aspect, a composition for reducing sebum production, comprising a sebum production inhibiting effective amount of a FAS inhibitor and a cosmetically acceptable carrier. FAS inhibitor comprises about 0.0001% to about 50% of the composition. The inventive compositions may additionally include another skin benefit agent. In another aspect, the present invention is a cosmetic system for reducing sebum production in skin, comprising said cosmetic composition a container for said composition, and instructions for applying said composition to said skin.

In another aspect, the invention provides a method of making a cosmetic composition for reducing sebum production in the sebocyte, comprising combining a sebum production reducing effective amount of a FAS inhibitor with a cosmetically acceptable carrier.

The FAS inhibitors are triclosan, cerulenin, EGCG, α-methylene-γ-butyrolactone, mixtures thereof, and analogs thereof, as well as cosmetically acceptable salts or solvates thereof.

The invention further relates to a cosmetic method of identifying sebum production inhibiting actives comprising contacting the active with FAS and detecting a reduction in sebum amount production in sebocytes.

### DETAILED DESCRIPTION OF THE INVENTION

The invention provides methods and cosmetic compositions for reducing sebum production using agents that inhibit the activity of fatty acid synthase (FAS). The present invention takes advantage of the discovery that the inhibition of the activity of FAS results in a decrease in sebum production in sebocytes. However, heretofore it was not known that the fatty acid biosynthetic pathway could affect sebogenesis.

Accordingly, in one aspect, the invention provides a method of decreasing sebum production in a sebocyte via the inhibition of mammalian FAS in the sebocyte or the skin of an individual. The method comprises contacting the sebocyte with a sebum production inhibiting amount of mammalian FAS inhibitor, thereby reducing sebum synthesis in the sebocyte.

In some embodiments, the FAS inhibitor is selected from the group consisting of cerulenin and a cerulenin analog, including pharmaceutically acceptable salts and solvates thereof.

In other embodiments, the FAS inhibitor is triclosan or analogs thereof. Triclosan is known to inhibit enoyl-reductase of type I fatty acid synthase.

In a further embodiment, the FAS inhibitor is EGCG, a polyphenolic compound that is a green tea extract.

In a still further embodiment, the mammalian FAS inhibitor is α-methylene-γ-butyrolactone.

As used herein, the term “comprising” means including, made up of, composed of, consisting and/or consisting essentially of. Except in the operating and comparative examples, or where otherwise explicitly indicated, all numbers in this description indicating amounts or ratios of material or conditions of reaction, physical properties of materials and/or use are to be understood as modified by the word “about”.

The term “about” is used herein to mean approximately, in the region of, roughly, or around. When the term “about” is used in conjunction with a numerical range, it modifies that range by extending the boundaries above and below the numerical values set forth.

The phrase “inhibiting the activity of FAS” as used herein refers to about 10% to about 100% decrease in FAS activity. More preferably, the term “inhibiting the activity of FAS” refers to about a 25% to about a 100% decrease in FAS activity, and most preferably, to about a 50% to about a 100% decrease in FAS activity. The invention contemplates the inhibition of FAS via any of the aforementioned seven enzymatic steps required for FAS activity and any inherent steps or processes. A decrease or change in FAS activity can be measured by any method known to one skilled in the art.

“Inhibitors of FAS” include competitive and noncompetitive FAS inhibitors. A competitive FAS inhibitor is a molecule that binds the FAS enzyme in a manner that is mutually exclusive of substrate binding. Typically, a competitive inhibitor of FAS will bind to the active site. A non-competitive FAS inhibitor can be one which inhibits the synthesis of FAS, but its binding to the enzyme is not mutually exclusive over substrate binding. FAS inhibitors contemplated by this invention are compounds that reduce the activity of FAS in animal cells without any significant effect on other cellular activities, at least at comparable concentrations.

The term “reducing the appearance of oily or greasy skin” is meant herein to refer to any detectable reduction in skin sebum, e.g., a reduction visible to the naked eye, that occurs after contacting the skin of an individual with a treatment regimen comprising an inhibitor of FAS.

The term “reducing sebum production” is used herein to mean a detectable lowering of the amount of sebum synthesized by a sebocyte exposed to a compound that inhibits FAS as compared to the amount of sebum synthesized in the absence of such an inhibiting compound. The term “reduction” as used herein in relation to sebum and/or sweat means the complete prevention, control of secretion, or a degree of reduction of the formation of sebum and/or sweat, respectively. The term “lowering” preferably refers to 5 about a 10% to about a 100% decrease in the amount of sebum thereby synthesized. More preferably, the term “lowering” refers to about a 25% to about a 100% decrease in the amount of sebum synthesized. Most preferably, the term “lowering” refers to about a 50% to about a 100% decrease in the amount of sebum synthesized. The terms lowering, reducing, decreasing, suppressing and inhibiting when used in relation to sebum production are intended to be used interchangeably.

As used herein, the term “a person in need thereof” refers to an individual with a normal but noticeable and undesired oily skin condition or an individual that elects to

decrease sebum in the absence of a noticeable and undesired oily skin condition.

As used herein, a “sebum reducing effective amount” of a compound means an amount of the compound that detectably reducing sebum production in skin after a cosmetically effective period of time. One skilled in the art is able to determine a “cosmetically effective period time” based on the particular skin oil reducing effect desired.

The term “skin” as used herein includes the skin on or in the face, mouth, neck, chest, back, arms, hands, legs, and scalp.

Preferably, the methods and compositions of the invention are for application to a vertebrate cell or individual, more particularly to a mammalian cell or individual, and most preferably to a human cell or individual. The term “individual” is used is herein to refer to a vertebrate, a mammal or a human.

### FAS Inhibitors

A wide variety of compounds have been shown to inhibit FAS, and selection of a suitable FAS inhibitor for use in this invention is within the skill of the ordinary worker in this art. Compounds that inhibit FAS can be identified by testing the ability of a compound to inhibit fatty acid synthase activity. FAS inhibitors are exemplified in, for example, International Patent Publication WO 94/02108 to The Johns Hopkins University.

By way of non-limiting example, cerulenin is one non-competitive FAS inhibitor useful in the methods of the invention. Structurally, cerulenin is characterized as [2R,3S]-2,3-epoxy4-oxo-7,10-trans, trans-dodecanoic acid amide. Cerulenin was originally isolated as a potential antifungal antibiotic from the culture broth of Cephalosporium caerulens. Its mechanism of action has been shown to be inhibition, through irreversible binding, of β-ketoacyl-ACP synthase, the condensing enzyme required for the biosynthesis of fatty acids.

In some embodiments, the FAS inhibitor is selected from the group consisting of cerulenin and a cerulenin analog, including cosmetically acceptable salts and solvates thereof. As used herein, the term “analog” refers to a chemical compound that is structurally related to cerulenin and retains at least a measurable amount of FAS inhibitory activity. Non-limiting examples of cerulenin and cerulenein analogs include those described in U.S. Pat. No. 5,539,132 to Royer et al. Alternatively, cerulenin may be obtained commercially from Sigma (St. Louis, Mo.).

In other embodiments, the FAS inhibitor is triclosan or analogs thereof. Triclosan is known to inhibit enoyl-reductase of type I fatty acid synthase. In other embodiments, the inhibitors of FAS are 4-phenyl-5-phenylimino-[1,2,4] dithiazolidin-3-one) or 5-chloro 4-phenyl-[1,2]-dithiol-3-one).

In further embodiment, the FAS inhibitor is epigallocatechingallate (EGCG), a green tea extract, a polyphenolic compound. EGCG is available from Sigma Corp., St. Louis, Mo.

In a still further embodiment, the mammalian FAS inhibitor is α-methylene-γ-butyrolactone.

### Identifying Sebum Production Inhibitors

To determine if a FAS inhibitor is useful in the methods of the invention, any assay known to those with skill in the art which can demonstrate a reduction in sebum production may be used. For example, cultured sebocytes can be incubated with a proposed FAS inhibitor test compound and tested for sebum content, e.g., as described in Example 1 below. This sebum content is then compared with the sebum content of untreated, cultured sebocytes to determine if the FAS inhibitor inhibits sebum production.

In a non-limiting example, sebum production may be assayed in human primary sebocytes according to the following protocol.

### EXAMPLE 1

This example shows FAS expression in sebocytes and a decrease in sebum production in sebocytes with application of FAS inhibitors.

A. FAS Expression in Sebocytes

In part A of the experiment, it has been determined that the FAS gene and protein are both expressed in cultured sebocytes and are present during cell growth and differentiation. Expression level was measured in terms of intensity of signal on a Western blot and recorded in Fluorescence Units. In sebocytes, the expression appears to peak at or just prior to confluence, as shown in the Table below, in terms of percent expression relative to peak expression at different time points during proliferation and differentiation of cultured sebocytes.

TABLE 1

| Sebocyte confluence | Relative expression |
|---|---|
| level | level |
| 60% | 74 ± 4% |
| 80% | 94 ± 9% |
| 100% | 87 ± 18% |
| 1d post confluence | 62 ± 25% |
| 2d post confluence | 5 ± 6% |

As can be seen from the Table above, the FAS expression level reaches a maximum level in proliferating cells and then declines.

B. Fatty Acid Synthase Inhibitors

Part B of the experiment shows the percent reduction in total lipids produced by sebocytes in culture relative to a control sample which has only been incubated with vehicle. Cerulenin and EGCG ((−)-epigallocatechin gallate from green tea) have been cited in the scientific literature as being mammalian FAS inhibitors. Triclosan is known to inhibit the microbial enzyme which performs one of the reactions also carried out by the mammalian enzyme present in sebocytes. Another FAS inhibitor is alpha-methylene-gamma-butyrolactone.

In Vitro Sebocyte Lipogenesis Assay

Human sebaceous glands were isolated from the nose of a male (age 60) and cultured using submerged tissue culture techniques (Bajor et al, J. Invest. Dermatol., 102:1994, p. 564). These sebocytes accumulate intracellular lipid droplets characteristic of mature human sebum.

Harvested and passaged sebocytes were added to each well of a 96 well tissue culture plate and incubated at 37° C. in-the presence of 7.5% CO₂ for 10 days. The growth medium was changed three times per week. On the day of experimentation, the growth medium was removed and the sebocytes washed three times with phosphate buffered saline (PBS). Fresh phenol-free sebocyte growth media in 0.2 ml amount containing various concentrations of active agent speculated to inhibit lipogenesis was added to each well. Triplicate wells were utilized for each sample. Controls consisted of PBS, dimethyl sulfoxide (DMSO) or ethanol used to solubilize the lipophilic compounds, and phenol red, a compound which possesses estrogen-like activity. The cultures were incubated at 37° C./7.5% CO₂ for 24 hours. Radioactive label was prepared by adding 100 μl (micro-liters) of 14-C labelled acetic acid (Amersham, sodium salt, specific activity of 56 mCi/mmol) to 10 ml of 50 mM sodium acetate buffer. Then 50 μl was added to each well containing the sebocytes and active agents. The cultures were returned to the incubator for 4 hours. Thereafter the treatments and label were removed and the sebocytes rinsed three times with fresh PBS. Samples containing the 14-C label were extracted and the label counted using a Beckman scintillation counter. Triplicates were performed for each sample.

For each 96 well tissue culture plate, 20 samples could be analyzed. Of these, 1 sample was reserved for media, 1 sample for DMSO or ethanol, and 1 sample for phenol red leaving 17 remaining samples.

The results are shown in the table below:

TABLE 2

| | | % decrease |
|---|---|---|
| Inhibitor | Concentration | in lipid syn. |
| Cerulenin | 40 uM | 99.0 |
| 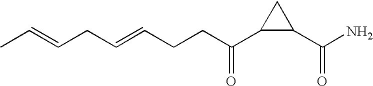 | 10 uM 5 uM 1.6 uM | 96.5 96.5 52.0 13.6 |
| Triclosan | 100 uM | 97.2 |
| 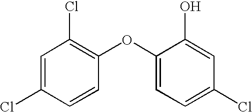 | 40 uM 20 uM | 90 48.8 |
| EGCG | 1000 uM | 90 |
| 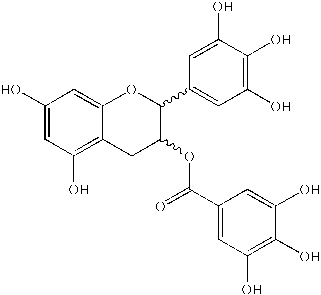 | 200 uM 40 uM 8 uM | 29.5 13.4 8.9 |
| α-methylene- | 500 uM | 100 |
| γ-butyrolactone | 100 uM | 53.3 |
| 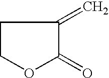 | 20 uM 10 uM | 13.2 1.3 |

Based on the results in the Table above, it is evident that cerulenin, triclosan, α-methylene γ-butyrolactone and EGCG have significant activity in reducing lipid synthesis in sebocytes, which would result in reduction of oiliness and grease in skin. Thiolactomycin, a compound that specifically inhibits the same reaction as cerulenin in microbial systems, was found not to inhibit fatty acid synthesis in sebocytes, exemplifying the specificity of our assay for mammalian FAS activity. It was found that γ-methylene γ-butyrolactone is also ineffective at reducing lipid synthesis.

While the FAS inhibitors discussed herein are typically small molecule compounds that directly inhibit the enzyme, it will be readily apparent to one skilled in the art that specific prevention of FAS biosynthesis is an equivalent procedure which will accomplish the same desired result. Therefore, this invention also contemplates inhibition of FAS biosynthesis as a method for inhibiting sebum production. This may be accomplished by selectively degrading mRNA encoding FAS or otherwise interfering with its transcription and/or translation. This may be accomplished, for instance, by introduction of a ribozyme specific for FAS mRNA or by antisense RNA complementary to the nucleic acid sequence of FAS. The sequence of human FAS cDNA is deposited with GenBank (GenBank No. 004104). In one embodiment, antisense therapy involves an expression vector containing at least a portion of the sequence encoding human FAS operably linked to a promoter such that it will be expressed in antisense orientation. In another embodiment, an antisense sequence is designed with the knowledge of the known FAS sequence and synthesized chemically. In either case, the antisense molecules may be produced in a fashion in which the molecules are degradation resistant. Such modifications include a modified backbone, a cap structure, or any other modification known to those in the art that prevents degradation. As a result, antisense molecules complementary to and capable of binding or hybridizing to FAS mRNA will be produced. Upon binding to FAS mRNA, translation of that mRNA is prevented, and consequently FAS is not produced. Production and use of antisense expression vectors is described in more detail in U.S. Pat. No. 5,107,065 to Shewmaker et al. and U.S. Pat. No. 5,190,931 to Inouye.

Cosmetic Methods and Compositions for Reducing Sebum Production

The inhibition of FAS in sebocytes, and the subsequent inhibition or decrease in sebum synthesis that occurs as a result thereof, are useful in methods designed to reduce sebum production in the skin. Thus, contact of sebocytes, either in vitro or in vivo, with an amount of an inhibitor of FAS that is effective to inhibit sebum production, will result in the desired sebum reducing effect. Suitable compounds for this purpose include those described above for inhibiting FAS activity and include, but are not limited to, cerulenin and FAS-inhibitory analogs thereof, triclosan and analogs thereof, α-methylene-γ-butyrolactone and FAS-inhibitory analogs thereof, and EGCG.

For cosmetic uses, it is preferred that a compound that reduces skin sebum by inhibiting the activity of FAS in a sebocyte of the skin be part of a cosmetic composition. Cosmetic compositions of the invention may be administered to a human or animal having a condition that normally occurs in skin, of a type that causes over-production of sebum.

The compositions and methods of the current invention are useful for cosmetic purposes. For example, occurrences in the skin or hair of noticeable but undesired feel or appearance of oiliness as a result of sebum production or overproduction may be ameliorated using the methods of the present invention. Cosmetic applications for methods of the present invention include the application of compositions containing one or more compounds that decreases sebum production in a sebocyte by inhibiting FAS in the sebocyte to enhance or otherwise alter the visual appearance of skin or hair. Alternatively, the prevention of sebum production, for example as a result of sweating or perspiration, is also contemplated as an appropriate application of the cosmetic methods of the invention for reducing the appearance of oily skin.

FAS inhibitor compounds are used in the inventive compositions in amounts of about 0.00001 to about 50%, preferably about 0.1 to about 20%, more preferably about 1 to about 15%, most preferably about 2 to about 5%.

Optional Skin Benefit Agents

Various types of additional active ingredients may be present in cosmetic compositions of the present invention. Actives are defined as skin benefit agents other than emollients and other than ingredients that merely improve the physical characteristics of the composition. Although not limited to this category, general examples include additional anti-sebum ingredients such as talcs and silicas, as well as alpha-hydroxy acids, beta-hydroxy acids, poly-hydroxy acids, benzoyl peroxide, astringent salts such as zinc salts, retinoids, sunscreens, and preservatives.

Beta-hydroxy acids include salicylic acid, for example. Zinc pyrithione is an example of zinc salts useful in the compositions of the present invention.

Optionally, but preferably, astringent salts are included in the compositions of the present invention. The astringent salts may be inorganic or organic salts of aluminum, zirconium, zinc and mixtures thereof. Preferably, the astringent salts are employed herein in particulate form, i.e., hydrophilic porous particles, of less than about 100 microns in size, preferably about 3 microns to about 10 microns in size. Salts useful as astringents or as components of astringent aluminum complexes include aluminum hydroxide, aluminum halides, aluminum hydroxyhalides, zirconyl oxyhalides, zirconyl hydroxyhalides and mixtures of these salt materials.

Aluminum salts of this type include aluminum chloride and the aluminum hydroxyhalides having the general formula Al₂(OH)ₓQ_{y}-XH₂O where Q is chlorine, bromine or iodine, where x is 2 to 5 and x+y=6 and x and y do not need to be integers; and where X is about 1 to 6. For example, aluminum chlorohydrate, having the formula [Al₂(OH)₅Cl]—XH₂O, is preferred, due to its ready commercial availability and relatively low cost.

Several types of complexes utilizing the above astringent salts are known in the antiperspirant art. For example, U.S. Pat. No. 3,792,068 (Luedders et al.), discloses complexes of aluminum, zirconium and amino acids such as glycine. Complexes reported therein and similar structures are commonly known as ZAG. The ZAG complexes ordinarily have an Al:Zr ratio of from about 1.67 to 12.5 and a Metal:Cl ratio of from about 0.73 to 1.93. The preferred amino acid for preparing such ZAG-type complexes is glycine of the formula CH₂(NH₂)COOH. Spherical ZAG, with particle size 1 to 100 microns, is especially preferred.

More specifically, the following is a list of astringent salts which may be useful for the present invention and which have approved listings under the United States Food & Drug Administration, Federal Register. They include aluminum chloride, aluminum chlorohydrate, aluminum chlorohydrex, aluminum chlorohydrex PEG, aluminum chlorohydrex PG, aluminum dichlorohydrate, aluminum dichlorohydrex PEG, aluminum dichlorohydrex PG, aluminum sesquichlorohydrate, aluminum sesquichlorohydrex PEG, aluminum sesquichlorohydrex PG, aluminum sulfate, aluminum zirconium octachlorohydrate, aluminum zirconium octachlorohydrex GLY (abbreviation for glycine), aluminum zirconium pentachlorohydrate, aluminum zirconium pentachlorohydrex GLY, aluminum zirconium tetrachlorohydrate, aluminum zirconium trichlorohydrate, aluminum zirconium tetrachlorohydrate GLY, and aluminum zirconium trichlorohydrate GLY.

Also suitable are:
potassium aluminium sulphate, also known as alum (KAI(SO₄)₂12H₂O),
aluminium undecylenoyl collagen amino acid,
sodium aluminium lactate+aluminium sulphate Al₂(SO₄)₃+Na₂HAl(OOCCHOHCH₃)₂—(OH)₆),
sodium aluminium chlorohydroxylactate,
aluminium bromohydrate (Al₂Br(OH)₅nH₂O),
aluminium chloride (AlCl₃6H₂O),
complexes of zinc salt and of sodium salt,
complexes of lanthanum and cerium, and
the aluminium salt of lipoamino acids (R—CO—NH—CHR′—CO—OAl—(OH)₂ with R=C₆/C₁₁ and R′=amino acid).

Preferably, the antiperspirant is an aluminium salt and, more preferably, it is chosen from potassium aluminium sulphate (alum) and aluminium chlorohydrate.

Amounts of the active astringent salt may range from about 0.000001% to about 20%, preferably from about 0.10% to about 18%, more preferably about 1 to about 15%, and optimally about 2% to about 3% by weight of the composition.

Aluminum chlorohydrate, referred to herein in shortened form as ACH, is the most preferred astringent salt for the purposes of the present invention, due to its wide commercial availability and relatively low cost.

Optionally, but preferably, the inventive compositions may also include a retinoid. Retinoids increase collagen synthesis by dermal fibroblasts. This results in smoothening of wrinkled skin. Addition of retinoids also provides improved inhibition of lipogenesis. The term “retinoids” as used herein includes retinoic acid, retinol, retinal, and retinyl esters. Included in the term “retinoic acid” are 13-cis retinoic acid and all-trans retinoic acid.

The term “retinol” as used herein includes the following isomers of retinol: all-trans-retinol, 13-cis-retinol, 11-cis-retinol, 9-cis-retinol, 3,4-didehydro-retinol. Preferred isomers are all-trans-retinol, 13-cis-retinol, 3,4-didehydro-retinol, 9-cis-retinol, 9-cis-retinol. Most preferred is all-trans-retinol, due to its wide commercial activity.

Retinyl ester is an ester of retinol. The term “retinol’ has been defined above. Retinyl esters suitable for use in the present invention are C₁-C₃₀ esters of retinol, preferably C₂-C₂₀ esters, and most preferably C₂, C₃, and C₁₆ esters because they are more commonly available. Examples of retinyl esters include but are not limited to: retinyl palmitate, retinyl formate, retinyl acetate, retinyl propionate, retinyl butyrate, retinyl valerate, retinyl isovalerate, retinyl hexanoate, retinyl heptanoate, retinyl octanoate, retinyl nonanoate, retinyl decanoate, retinyl undecanoate, retinyl laurate, retinyl tridecanoate, retinyl myristate, retinyl pentadecanoate, retinyl heptadecanoate, retinyl stearate, retinyl isostearate, retinyl nonadecanoate, retinyl arachidonate, retinyl behenate, retinyl linoleate, retinyl oleate, retinyl lactate, retinyl glycolate, retinyl hydroxy caprylate, retinyl hydroxy laurate, retinyl tartarate.

The retinoids in the present invention are present in an amount of from 0.001% to 10%, preferably from 0.01% to 1%, and most preferably from 0.01% to 0.05%.

Sunscreens include those materials commonly employed to block ultraviolet light. Illustrative compounds are the derivatives of PABA, cinnamate and salicylate. For example, avobenzophenone (Parsol 1789®) octyl methoxycinnamate and 2-hydroxy-4-methoxy benzophenone (also known as oxybenzone) can be used. Octyl methoxycinnamate and 2-hydroxy-4-methoxy benzophenone are commercially available under the trademarks, Parsol MCX and Benzophenone-3, respectively. The exact amount of sunscreen employed in the compositions can vary depending upon the degree of protection desired from the sun's UV radiation.

Many cosmetic compositions, especially those containing water, must be protected against the growth of potentially harmful microorganisms. Suitable preservatives include alkyl esters of p-hydroxybenzoic acid, hydantoin derivatives, propionate salts, and a variety of quaternary ammonium compounds. Particularly preferred preservatives of this invention are methyl paraben, propyl paraben, phenoxyethanol and benzyl alcohol. Preservatives will usually be employed in amounts ranging from about 0.1% to 2% by weight of the composition.

Cosmetically Acceptable Vehicle

The compositions according to the invention comprise a cosmetically acceptable vehicle to act as a diluant, dispersant or carrier of FAS inhibitor compounds and any optional skin benefit agents, so as to facilitate their distribution when the composition is applied to the skin.

The vehicle may be aqueous, anhydrous or an emulsion. Preferably, the compositions are aqueous or an emulsion, especially water-in-oil or oil-in-water emulsion. Water when present will be in amounts which may range from 5 to 99%, preferably from 40 to 90%, optimally between 60 and 90% by weight.

Besides water, relatively volatile solvents may also serve as carriers within compositions of the present invention. Most preferred are monohydric C₁-C₃ alkanols. These include ethyl alcohol, methyl alcohol and isopropyl alcohol. The amount of monohydric alkanol may range from 1 to 70%, preferably from 10 to 50%, optimally between 15 and 40% by weight.

Emollient materials may also serve as cosmetically acceptable carriers. These may be in the form of silicone oils and synthetic esters. Amounts of the emollients may range anywhere from 0.1 to 50%, preferably between 1 and 20% by weight.

Silicone oils may be divided into the volatile and non-volatile variety. The term “volatile” as used herein refers to those materials which have a measurable vapor pressure at ambient temperature. Volatile silicone oils are preferably chosen from cyclic or linear polydimethylsiloxanes containing from 3 to 9, preferably from 4 to 5, silicon atoms. Linear volatile silicone materials generally have viscosities less than about 5 centistokes at 25° C. while cyclic materials typically have viscosities of less than about 10 centistokes. Nonvolatile silicone oils useful as an emollient material include polyalkyl siloxanes, polyalkylaryl siloxanes and polyether siloxane copolymers. The essentially non-volatile polyalkyl siloxanes useful herein include, for example, polydimethyl siloxanes with viscosities of from about 5 to about 25 million centistokes at 25° C. Among the preferred non-volatile emollients useful in the present compositions are the polydimethyl siloxanes having viscosities from about 10 to about 400 centistokes at 25° C.

Among the ester emollients are:
(1) Alkenyl or alkyl esters of fatty acids having 10 to 20 carbon atoms. Examples thereof include isoarachidyl neopentanoate, isononyl isonanonoate, oleyl myristate, oleyl stearate, and oleyl oleate.
(2) Ether-esters such as fatty acid esters of ethoxylated fatty alcohols.
(3) Polyhydric alcohol esters. Ethylene glycol mono and di-fatty acid esters, diethylene glycol mono- and di-fatty acid esters, polyethylene glycol (200-6000) mono- and di-fatty acid esters, propylene glycol mono- and di-fatty acid esters, polypropylene glycol 2000 monooleate, polypropylene glycol 2000 monostearate, ethoxylated propylene glycol monostearate, glyceryl mono- and di-fatty acid esters, polyglycerol poly-fatty esters, ethoxylated glyceryl monostearate, 1,3-butylene glycol monostearate, 1,3-butylene glycol distearate, polyoxyethylene polyol fatty acid ester, sorbitan fatty acid esters, and polyoxyethylene sorbitan fatty acid esters are satisfactory polyhydric alcohol esters.
(4) Wax esters such as beeswax, spermaceti, myristyl myristate, stearyl stearate and arachidyl behenate.
(5) Sterols esters, of which cholesterol fatty acid esters are examples.

Fatty acids having from 10 to 30 carbon atoms may also be included as cosmetically acceptable carriers for compositions of this invention. Illustrative of this category are pelargonic, lauric, myristic, palmitic, stearic, isostearic, hydroxystearic, oleic, linoleic, ricinoleic, arachidic, behenic and erucic acids.

Humectants of the polyhydric alcohol type may also be employed as cosmetically acceptable carriers in compositions of this invention. The humectant aids in increasing the effectiveness of the emollient, reduces scaling, stimulates removal of built-up scale and improves skin feel. Typical polyhydric alcohols include glycerol, polyalkylene glycols and more preferably alkylene polyols and their derivatives, including propylene glycol, dipropylene glycol, polypropylene glycol, polyethylene glycol and derivatives thereof, sorbitol, hydroxypropyl sorbitol, hexylene glycol, 1,3-butylene glycol, 1,2,6-hexanetriol, ethoxylated glycerol, propoxylated glycerol and mixtures thereof. For best results the humectant is preferably propylene glycol or sodium hyaluronate. The amount of humectant may range anywhere from 0.5 to 30%, preferably between 1 and 15% by weight of the composition.

Thickeners may also be utilized as part of the cosmetically acceptable carrier of compositions according to the present invention. Typical thickeners include crosslinked acrylates (e.g. Carbopol 982), hydrophobically-modified acrylates (e.g. Carbopol 1382), cellulosic derivatives and natural gums. Among useful cellulosic derivatives are sodium carboxymethylcellulose, hydroxypropyl methylcellulose, hydroxypropyl cellulose, hydroxyethyl cellulose, ethyl cellulose and hydroxymethyl cellulose. Natural gums suitable for the present invention include guar, xanthan, sclerotium, carrageenan, pectin and combinations of these gums. Amounts of the thickener may range from 0.0001 to 5%, usually from 0.001 to 1%, optimally from 0.01 to 0.5% by weight.

Collectively, the water, solvents, silicones, esters, fatty acids, humectants and/or thickeners will constitute the cosmetically acceptable carrier in amounts from 1 to 99.9%, preferably from 80 to 99% by weight.

An oil or oily material may be present, together with an emulsifier to provide either a water-in-oil emulsion or an oil-in-water emulsion, depending largely on the average hydrophilic-lipophilic balance (HLB) of the emulsifier employed.

Use of the Novel Compositions

The compositions according to the invention are intended primarily as a product for topical application to human skin, especially as an agent for controlling or preventing excessive sebum secretion. Suppression of sebum provides multiple benefits, including: improved skin condition; reduction of an unpleasant appearance and feel of greasy skin; reduction and/or prevention of acne, rosacea, seborrhea, oily scalp, oily/greasy hair, and dandruff.

In use, a quantity of the composition, for example from 1 to 100 ml, is applied to exposed areas of the skin, from a suitable container or applicator and, if necessary, it is then spread over and/or rubbed into the skin using the hand or fingers or a suitable device.

The present invention also includes a cosmetic method of controlling or preventing an oily skin condition, especially in the facial area, by applying to the skin the inventive composition. In another aspect, the present invention includes a cosmetic method of controlling, preventing, or treating oily or greasy hair.

The invention also includes a cosmetic method of reducing, preventing or controlling sebum secretion from sebocytes by applying the inventive composition.

The invention also includes a cosmetic method of reducing or controlling the perception of oily or greasy skin by applying to the skin the inventive composition.

The inventive methods and compositions provide control of sebum secretion from sebocytes, improved oil control and improved skin feel, and prevent shine and stickiness, while also providing anti-microbial activity against bacteria associated with acne and, generally, controlling microbial activity of bacteria on the skin surface.

Product Form and Packaging

The cosmetic skin composition of the invention can be in any form, e.g. formulated as a toner, gel, lotion, a fluid cream, or a cream. The composition can be packaged in a suitable container to suit its viscosity and intended use by the consumer. For example, a lotion or fluid cream can be packaged in a bottle or a roll-ball applicator or a propellant-driven aerosol device or a container fitted with a pump suitable for finger operation. When the composition is a cream, it can simply be stored in a non-deformable bottle or squeeze container, such as a tube or a lidded jar. The invention accordingly also provides a closed container containing a cosmetically acceptable composition as herein defined.

The composition may also be included in capsules such as those described in U.S. Pat. No. 5,063,057.

The composition with the packaging, and together with a set of instructions associated with the package, may comprise a system for controlling skin sebum. The set of instructions may be printed on the package or placed in the package. The set of instructions typically communicates to the consumer of the present articles to dispense the composition in an amount effective to provide a solution to problems involving, and/or provision of a benefit relating to, those selected from the group consisting of killing or reducing the level of microorganisms, reducing sebum production, improving appearance, and/or a combination thereof. It is important that the consumer of the present article be aware of these benefits, since otherwise the consumer would not know that the composition would solve these problems or combination of problems and/or provide these benefits or combination of benefits.

While the present invention has been described herein with some specificity, and with reference to certain preferred embodiments thereof, those of ordinary skill in the art will recognize numerous variations, modifications and substitutions of that which has been described which can be made, and which are within the scope and spirit of the invention. It is intended that all of these modifications and variations be within the scope of the present invention as described and claimed herein, and that the inventions be limited only by the scope of the claims which follow, and that such claims be interpreted as broadly as is reasonable. Throughout this application, various publications have been cited. The entireties of each of these publications are hereby incorporated by reference herein.

1. A cosmetic method of decreasing sebum production in sebocytes or in skin of an individual in need thereof, comprising contacting said sebocyte or skin with sebum production inhibiting amount of a fatty acid synthase (FAS) inhibitor, thereby reducing sebum synthesis in said sebocyte or skin.

2. The cosmetic method according to claim 1, wherein said FAS inhibitor is selected from the group consisting of triclosan, cerulenin, EGCG, α-methylene-γ-butyrolactone, mixtures thereof, and analogs thereof.

3. The cosmetic method according to claim 1, wherein said method reduces synthesis of squalene, triglycerides, free fatty acids, wax esters, cholesterol, sterol esters, and/or a combination thereof.

4. The cosmetic method according to claim 2, wherein said FAS inhibitor is a cosmetically acceptable salt or solvate of said FAS inhibitor.

5. A cosmetic method of identifying sebum production inhibiting actives comprising contacting the active with FAS and detecting a reduction in sebum amount production in sebocytes.

6. A cosmetic composition for reducing sebum production, comprising a sebum production inhibiting effective amount of one or more FAS inhibitor(s) and a cosmetically acceptable carrier.

7. The cosmetic composition according to claim 6, wherein said FAS inhibitor is selected from the group consisting of triclosan, cerulinin, EGCG, α-methylene-γ-butyrolactone, mixtures thereof, and analogs thereof.

8. The cosmetic composition according to claim 6, wherein said composition is applied in a method that reduces synthesis of squalene, triglycerides, free fatty acids, wax esters, cholesterol, sterol esters, and/or a combination thereof.

9. The cosmetic composition according to claim 7, wherein said FAS inhibitor is a cosmetically acceptable salt or solvate of said FAS inhibitor.

10. The cosmetic composition according to claim 6, wherein said FAS inhibitor comprises about 0.0001% to about 50% of said composition.

11. The cosmetic composition according to claim 6, further comprising a skin benefit agent.

12. A method of making a cosmetic composition for reducing sebum production in the sebocyte according to claim 6, comprising combining said sebum production reducing effective amount of a FAS inhibitor with a cosmetically acceptable carrier.

13. A cosmetic system for reducing sebum production in skin, comprising said cosmetic composition according to claim 6, a container for said composition, and instructions for applying said composition to said skin.
